(19) United States
(12) Patent Application Publication
(10) Pub. No.: US 2014/0336490 A1
(43) Pub. Date: Nov. 13, 2014

(54) ACCESSORY DEVICE FOR TWELVE-LEAD ELECTROCARDIOGRAPHY APPARATUS

(71) Applicant: Wei-Chun HUANG, Kaohsiung City (TW)

(72) Inventor: Wei-Chun HUANG, Kaohsiung City (TW)

(21) Appl. No.: 14/065,935

(22) Filed: Oct. 29, 2013

(30) Foreign Application Priority Data
May 10, 2013 (TW) 102208731

(51) Int. Cl. A61B 5/0408

(52) U.S. Cl. CPC A61B 5/04085
USPC 600/382

(57) ABSTRACT

An accessory device for a twelve-lead electrocardiography (ECG) apparatus includes a blanket. The ECG apparatus includes an ECG machine that has ten first electrical couplers, and ten electrodes each having a second electrical coupler. The blanket includes a sheet body configured to cover the body of a patient and formed with ten holes that are arranged according to a standard electrode placement for twelve-lead ECG measurement and that are configured to permit the second electrical couplers of the ten electrodes to extend therethrough, respectively.

### CROSS-REFERENCE TO RELATED APPLICATION

This application claims priority of Taiwanese patent application no.102208731, filed on May 10, 2013.

### BACKGROUND OF THE INVENTION

1. Field of the Invention

The present invention relates to an accessory device for a twelve-lead electrocardiography (ECG) apparatus.

2. Description of the Related Art

Electrocardiography (ECG) is a method for rapid detection of acute myocardial infarction. Currently, the twelve lead ECG (lead) is the international standard, including six leads on the front chest and four leads on the limbs. When performing electrocardiography, a plurality of electrodes is to be disposed in correct positions on the subject's body in order to obtain the correct measurement result.

Referring to FIG. 1, Taiwanese Utility Model No. M403996 discloses an ECG blanket 1, including a blanket body 11 for covering a subject 100, an L-shaped hole 12 formed in the blanket body 11, and an indicator strip 13 provided on the blanket body 11 and having a plurality of indicator lines 131. By the L-shaped hole 12, electrodes (not shown) can be quickly disposed on the subject 100, with reference to the indicator lines 131.

Although the L-shaped hole 12 is designed to reveal to an operator the setup positions of the electrodes on the subject 100 and to preserve body temperature and protect privacy of the subject 100, the blanket body 11 lacks clear marks for alignment of different body sizes of different subjects 100 with the L-shaped hole 12, creating the possibility of misalignment.

Moreover, while the blanket body 11 has a plurality of indicator lines 131, it does not indicate the precise positions for placement of the electrodes, slowing down the electrocardiography procedure. For subject 100 with acute chest pain, such delay in time may be highly disadvantageous for the subsequent therapy.

### SUMMARY OF THE INVENTION

The object of the present invention is to provide an accessory device for a twelve-lead electrocardiography (ECG) apparatus that may facilitate twelve-lead ECG measurements.

The ECG apparatus includes an ECG machine that has ten first electrical couplers, and ten electrodes each having a second electrical coupler for coupling electrically and removably to one of the first electrical couplers. The electrodes are configured to attach to a body of a patient to conduct a twelve-lead ECG measurement.

The accessory device of this invention includes a blanket including a sheet body configured to cover the body of the patient and formed with ten holes. The ten holes are arranged according to a standard electrode placement for the twelve-lead ECG measurement and are configured to permit the second electrical couplers of the ten electrodes to extend therethrough, respectively.

When conducting the twelve-lead ECG measurement, the ten electrodes are disposed on one side of the sheet body with the second electrical couplers thereof extending through the ten holes respectively, and the first electrical couplers are disposed on an opposite side of the sheet body and are coupled electrically and respectively to the second electrical couplers.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic illustration illustrating an ECG blanket disclosed in Taiwanese Utility Model No. M403996 in a state of use;

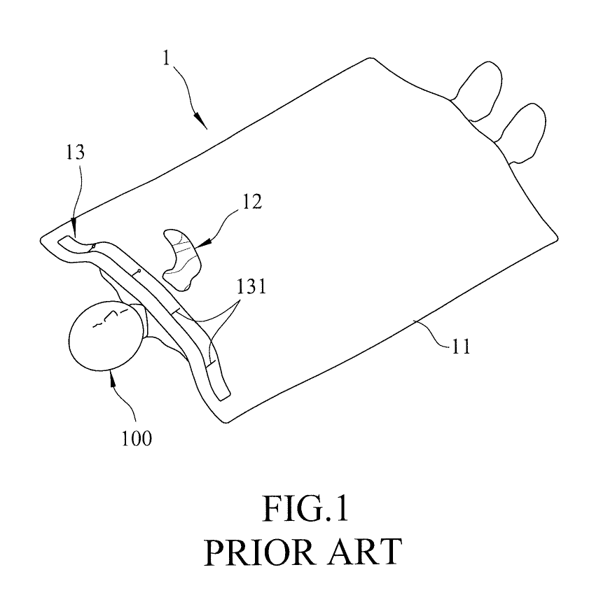

FIG. 2 is a plan view illustrating an accessory device for a twelve-lead electrocardiography (ECG) apparatus of a preferred embodiment of the present invention;

FIG. 3 is a schematic illustration of an ECG machine that has ten first electrical couplers and ten electrodes each having a second electrical coupler;

FIG. 4 is a side view illustrating the first electrical coupler electrically coupled with the second electrical coupler; and

FIG. 5 is a schematic illustration illustrating the preferred embodiment in use.

### DETAILED DESCRIPTION OF THE PREFERRED EMBODIMENT

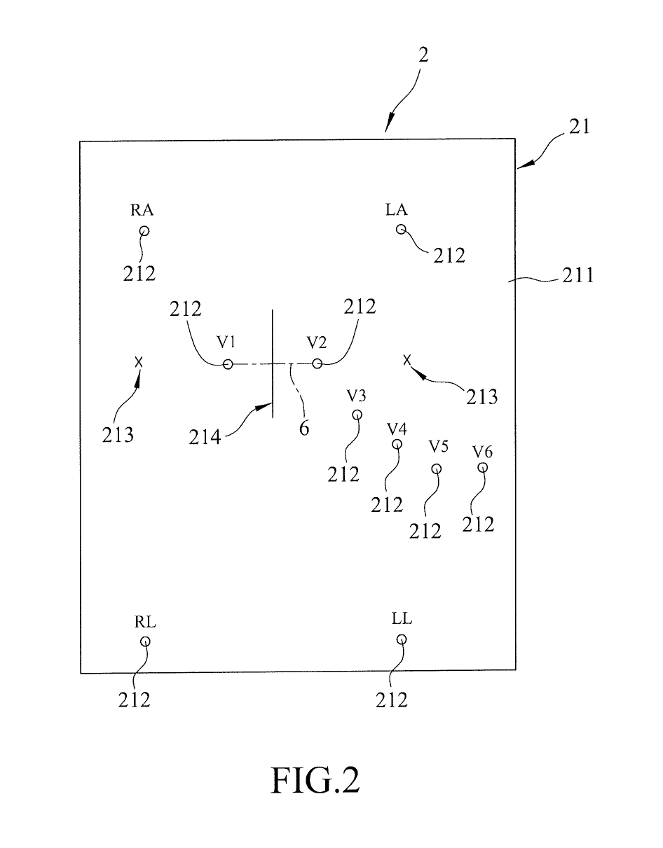

Referring to FIG. 2, a preferred embodiment of an accessory device 2 for a twelve-lead electrocardiography (ECG) apparatus includes a blanket 21. The blanket 21 includes a sheet body 211 formed with ten holes 212 that extend therethrough and that are arranged according to a standard electrode placement for the twelve-lead ECG measurement, two nipple position markers 213 formed on the sheet body 211, and a sternum midline position marker 214 disposed between the nipple position markers 213 and corresponding in position to a midline position of a sternum of a patient.

The holes 212 are based on the standard electrode placement for the twelve-lead ECG measurement, and are respectively labeled as V1, V2, V3, V4, V5, V6, RA (right arm), LA (left arm), RL (right leg) , and LL (left leg). The two nipple position markers 213 and holes 212 labeled as V1, V2 are positioned on the same horizontal line 6, and the sternum midline position marker 214 is positioned in between of the holes 212 labeled as V1, V2. The sternum midline position marker 214 is orthogonal to the line 6 that interconnects the holes 212. The two nipple position markers 213 may be labeled as “right nipple” and “left nipple”, and the sternum midline position marker 214 may be labeled as “sternum midline”. The table below describes the body locations that V1 to V6 correspond to respectively, and will not be described in great detail since such should be readily appreciated by those skilled in the art.

| | |
|---|---|
| V1 | In the fourth intercostal space on the right |
| | side of the chest (in between the fourth rib |
| | and the fifth rib on the right side of the chest) |
| V2 | In the fourth intercostal space on the left side |
| | of the chest (in between the fourth rib and the |
| | fifth rib on the left side of the chest) |
| V3 | In between V2 and V4 |
| V4 | In the fifth intercostal space on the left side |
| | of the chest (in between the fifth rib and the |
| | sixth rib on the left side of the chest) in the |
| | mid-clavicular line |
| V5 | In the fifth intercostal space on the left side |
| | of the chest and in the left anterior axillary |
| | line |
| V6 | In the fifth intercostal space on the left side |
| | of the chest and in the midaxillary line |

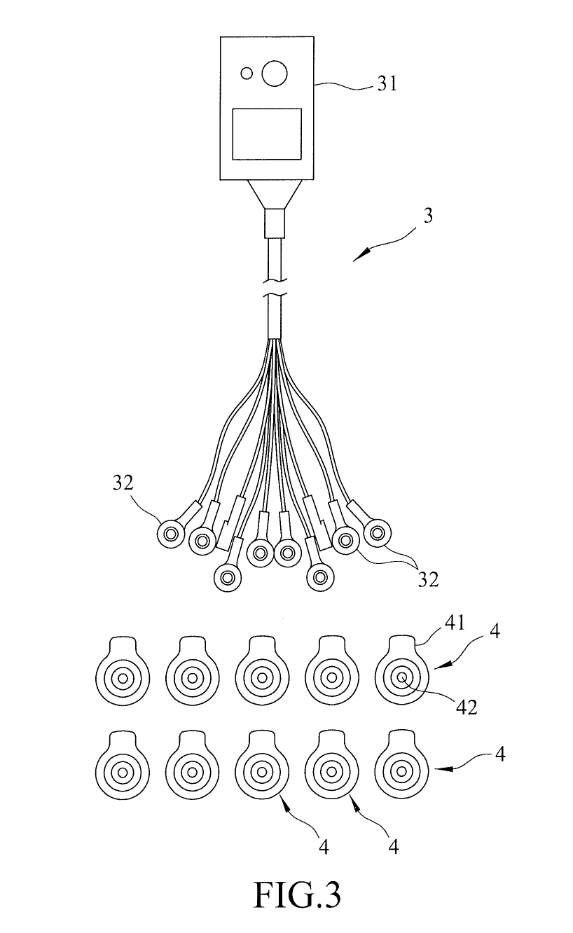

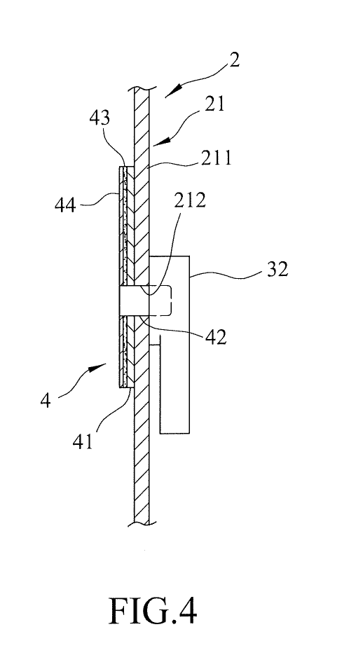

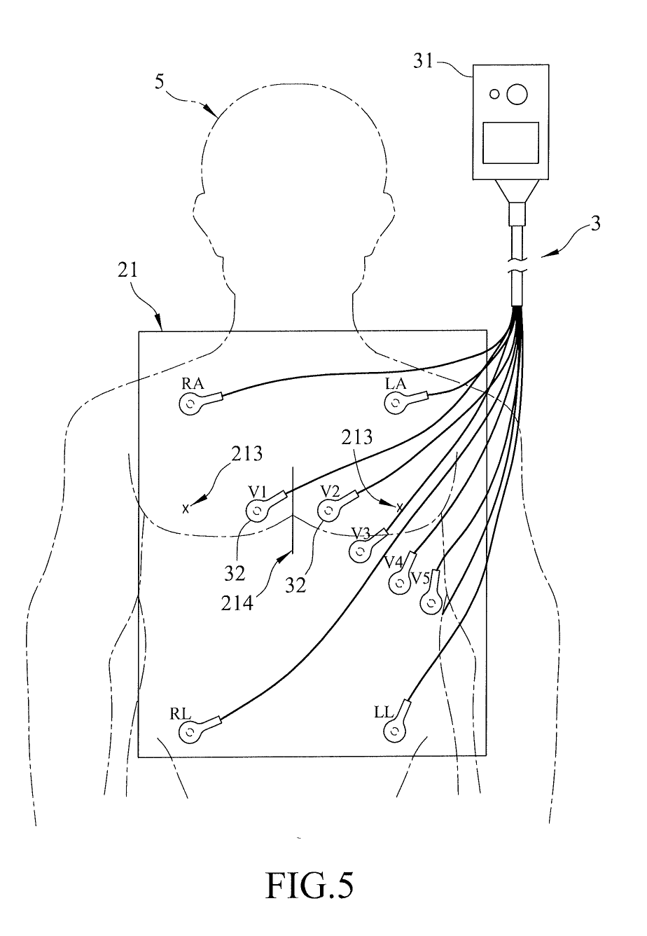

Referring to FIGS. 3 and 4, the auxiliary device 2 is to be used with an ECG apparatus 3 that has ten electrodes 4 for conducting a twelve-lead ECG measurement on a patient 5 (see FIG. 5). Referring to FIG. 3, the ECG apparatus 3 includes an ECG machine 31 that has ten first electrical couplers 32. Each electrode 4 includes an electrode body 41 and a second electrical couplers 42 extending through the electrode body 41 and detachably engaging the first electrical coupler 32. As shown in FIG. 4, each second electrical coupler 42 extends through a corresponding hole 212, and then engages the corresponding first electrical coupler 32. In this manner, the electrode body 41 and the first electrical coupler 31 are disposed on opposite sides of the sheet body 211 of the blanket 21. For clarity, FIG. 4 illustrates only one electrode 4 and one first electrical coupler 32. Prior to use of the auxiliary apparatus 2, each of the electrodes 4 is connected with a corresponding one of the first electrical couplers 32 via a corresponding one of the holes 212 in order for the electrodes 4 and the first electrical couplers 32 to be disposed on the sheet body 211 of the blanket 21.

Referring to FIGS. 4 and 5, the blanket 21 is covered on a body of a patient 5 such that the two nipple position markers 213 correspond with the nipple positions of the patient 5 and the sternum midline position marker 214 corresponds with the midline position of a sternum of the patient 5. Due to different patients 5 having different widths between nipples, one has to only align the sternum midline position marker 214 to correspond with the midline position of a sternum of the patient 5 and align the two nipple position markers 213 with the line interconnecting the two nipples of the patient 5. After the alignment, the electrodes 4 can then be accurately attached to their corresponding positions on the patient 5.

Referring to FIG. 4, each electrode 4 is usually provided with an adhesive layer 43 on one side of the electrode body 41 and a release paper 44 disposed on the adhesive layer 43. In use, the release paper 44 is removed to allow the electrode 4 to be attached onto the patient 5 by the adhesive layer 43.

Referring to FIGS. 4 and 5, the two electrodes 4 at the holes 212 labeled RA (right arm) and LA (left arm) are usually attached to the body of the patient 5 first, the three electrodes 4 at the holes 212 labeled V1, V2, V3 are then attached, the three electrodes 4 at the holes labeled v4, v5, v6 are subsequently attached (V6 is not shown in FIG. 5 as the viewing angle is blocked by the arm of the patient 5), and finally, the two electrodes 4 at the holes 212 labeled RL (right leg) and LL (left leg) are attached. The twelve-lead ECG measurement can then be carried out after the ten electrodes 4 are attached properly. After use, the used electrodes 4 are detached from the first electrical couplers 32, and are replaced with ten new electrodes 4 for a new patient 5.

In summary, the present invention enables an operator to quickly and accurately attach the ten electrodes 4 onto a patient 5, on an average of 30 seconds for completing a twelve-lead ECG measurement. The present invention is especially useful when applied in emergency situations in an ambulance or emergency room where every second counts.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. An accessory device for a twelve-lead electrocardiography (ECG) apparatus, the ECG apparatus including an ECG machine that has ten first electrical couplers, and ten electrodes each having a second electrical coupler for coupling electrically and removably to one of the first electrical couplers, the electrodes being configured to attach to a body of a patient to conduct a twelve-lead ECG measurement, said accessory device comprising:
a blanket including a sheet body configured to cover the body of the patient and formed with ten holes that are arranged according to a standard electrode placement for the twelve-lead ECG measurement and that are configured to permit the second electrical couplers of the ten electrodes to extend therethrough, respectively;
whereby, when conducting the twelve-lead ECG measurement, the ten electrodes are disposed on one side of said sheet body with the second electrical couplers thereof extending through said ten holes respectively, and the first electrical couplers are disposed on an opposite side of said sheet body and are coupled electrically and respectively to the second electrical couplers.

2. The accessory device as claimed in claim 1, wherein said sheet body is further formed with two nipple position markers corresponding respectively in position to left and right nipples on the body of the patient, and a sternum midline position marker disposed between said nipple position markers and corresponding in position to a midline position of a sternum of the patient.

3. The accessory device as claimed in claim 2, wherein said ten holes are labeled as V1, V2, V3, V4, V5, V6, RA, LA, RL and LL, respectively.

4. The accessory device as claimed in claim 3, wherein said nipple position markers and said holes that are labeled as V1 and V2 are arranged on a same horizontal line.

5. accessory device as claimed in claim 4, wherein said sternum midline position marker is disposed between said holes that are labeled as V1 and V2 and is generally perpendicular to a line interconnecting said holes that are labeled as V1 and V2.
